(19) United States
(12) Patent Application Publication
(10) Pub. No.: US 2024/0398774 A1
(43) Pub. Date: Dec. 5, 2024

(54) METHODS OF TREATING INFLAMMATORY DISEASES WITH A SELECTIVE GLUCOCORTICOID RECEPTOR MODULATOR

(71) Applicant: ASTRAZENECA AB, Södertälje (SE)

(72) Inventors: Mary N BROWN, Waltham, MA (US); Philip AMBERY, Södertälje (SE); Christina KEEN, Södertälje (SE)

(21) Appl. No.: 18/693,356

(22) PCT Filed: Sep. 16, 2022
(86) PCT No.: PCT/EP2022/075749

§ 371 Date: Mar. 19, 2024

Related U.S. Application Data
(60) Provisional application No. 63/245,989, filed on Sep. 20, 2021.

(51) Int. Cl. A61K 31/4439; A61P 3/10; A61P 29/00

(52) U.S. Cl. CPC A61K 31/4439; A61P 3/10; A61P 29/00

(57) ABSTRACT

This disclosure relates to methods for treating a chronic inflammatory disease or disorder in a subject having type 2 diabetes mellitus or being predisposed to type 2 diabetes mellitus. This disclosure also relates to methods for preventing development of diabetes mellitus due to glucocorticoid-induced hyperglycemia or inhibiting progression of preexisting type 2 diabetes mellitus in a subject.

### BACKGROUND OF THE DISCLOSURE

### Field of the Disclosure

This disclosure relates to methods for treating a chronic inflammatory disease or disorder in a subject having type 2 diabetes mellitus or being predisposed to type 2 diabetes mellitus. This disclosure also relates to methods for preventing development of diabetes mellitus due to glucocorticoid-induced hyperglycemia or inhibiting progression of preexisting type 2 diabetes mellitus in a subject.

### Description of Related Art

Glucocorticoids (GCs) are effective anti-inflammatory drugs that are widely used in patients who have inflammatory and autoimmune diseases. For example, GSs have been used to treat both acute and chronic diseases, including rheumatoid arthritis, asthma, chronic obstructive pulmonary disease (“COPD”), osteoarthritis, rheumatic fever, allergic rhinitis, systemic lupus erythematosus, Crohn's disease, inflammatory bowel disease, ulcerative colitis, etc. Examples of GCs include dexamethasone, prednisone, and prednisolone.

Both the duration and dose of oral GC treatment are often limited by severe and sometimes irreversible side effects, such as osteoporosis, hyperglycemia, effects on glucose metabolism (diabetes mellitus), skin thinning, hypertension, glaucoma, muscle atrophy, Cushing's syndrome, fluid homeostasis, and psychosis (depression). These side effects, largely resulting from disruption of metabolic function, can particularly limit the use of GCs in a chronic setting. Considering significant prevalence of diabetes and prediabetes in overall population, an anti-inflammatory medication with no or reduced dysglycaemic effects would be of great benefit to patients requiring chronic GC treatment.

A range of compounds have been reported to have the beneficial effects of GCs but have not shown promise with respect to hyperglycemia. Still there still exists a need for alternative therapies that possess the beneficial effects of GCs, but with a reduced likelihood of side effects.

### SUMMARY OF THE DISCLOSURE

One aspect of the disclosure provides methods for treating a chronic inflammatory disease or disorder in a subject having type 2 diabetes mellitus or being predisposed to type 2 diabetes mellitus. Such methods include administering to the subject a therapeutically effective amount of 2,2-difluoro-N-[(1R,2S)-3-methyl-1-[1-(1-methyl-6-oxopyridin-3-yl) indazol-5-yl]oxy-1-phenylbutan-2-yl]propanamide (AZD9567), or a salt, hydrate, solvate, or prodrug thereof,
wherein the therapeutically effective amount of AZD9567 is sufficient to treat the chronic inflammatory disease or disorder in the subject, and
wherein the therapeutically effective amount of AZD9567 is sufficient to control glycaemia in the subject.

Another aspect of the disclosure provides methods of preventing development of diabetes mellitus due to glucocorticoid-induced hyperglycemia in a subject in need of a treatment for a chronic inflammatory disease or disorder. Such methods include:
administering to the subject a therapeutically effective amount of AZD9567, or a salt, hydrate, solvate, or prodrug thereof,
   wherein the therapeutically effective amount of AZD9567 is sufficient to control the glucocorticoid-induced hyperglycemia in the subject and prevent development of diabetes mellitus.

Another aspect of the disclosure provides methods of inhibiting progression of diabetes mellitus in a subject in need of a treatment for a chronic inflammatory disease or disorder and having type 2 diabetes mellitus. Such methods include: administering to the subject a therapeutically effective amount of AZD9567, or a salt, hydrate, solvate, or prodrug thereof,
wherein the therapeutically effective amount of AZD9567 is sufficient to control the glucocorticoid-induced hyperglycemia in the subject and inhibit progression of diabetes mellitus.

These and other features and advantages of the present invention will be more fully understood from the following detailed description taken together with the accompanying claims. It is noted that the scope of the claims is defined by the recitations therein and not by the specific discussion of features and advantages set forth in the present description.

### BRIEF DESCRIPTION OF THE DRAWINGS

The accompanying drawings are included to provide a further understanding of the compositions and methods of the disclosure, and are incorporated in and constitute a part of this specification. The drawings illustrate one or more embodiment(s) of the disclosure and, together with the description, serve to explain the principles and operation of the disclosure.

FIG. 1 is a schematic representation of the study provided in the Example.

FIG. 2A illustrates Continuous Glucose Monitoring (CGM) in Cohort 1 patients of the Example of the disclosure.

FIG. 2B illustrates CGM in Cohort 2 patients. Arrows indicate time of dosing of AZD9567 or prednisolone.

FIG. 3A illustrates the difference in mean daily glucose (top) and change from baseline in mean daily glucose (bottom) in Cohort 1 patients of the Example of the disclosure. FIG. 3B illustrates the difference in mean daily glucose (top) and change from baseline in mean daily glucose (bottom) in Cohort 2 patients.

FIG. 4A shows reduction of glucose area under the effect-time curve from time 0 to 4 hours (AUC₀₋₄ₕ), glucagon AUC₀₋₄ₕ and insulin AUC₀₋₄ₕ after Mixed Meal Tolerance Test (MMTT) at Day 4 in Cohort 1.

FIG. 4B illustrates reduction of glucose AUC₀₋₄ₕ after MMTT at Day 4 in Cohort 2.

FIG. 5 illustrates cortisol change in Cohort 1 patients of the Example of the disclosure.

FIG. 6 illustrates adrenocorticotropic hormone change from baseline in Cohort 1 patients of the Example of the disclosure.

### DETAILED DESCRIPTION OF THE DISCLOSURE

Before the disclosed processes and materials are described, it is to be understood that the aspects described herein are not limited to specific embodiments, and as such can, of course, vary. It is also to be understood that the terminology used herein is for the purpose of describing particular aspects only and, unless specifically defined herein, is not intended to be limiting.

In view of the present disclosure, the methods and compositions described herein can be configured by the person of ordinary skill in the art to meet the desired need. The present disclosure provides improvements in treatment of chronic inflammatory conditions. Such methods include administering to the subject a therapeutically effective amount of AZD9567, or a salt, hydrate, solvate, or prodrug thereof, sufficient to treat the chronic inflammatory disease or disorder and control glycaemia in the subject.

In certain embodiments of the methods of the disclosure, the chronic inflammatory disease or disorder is responsive to oral corticosteroids. For example, in certain embodiments, the chronic inflammatory disease or disorder requires medium to high daily dose of oral corticosteroids (e.g., ≥10 mg prednisone, or equivalent nominal dose for other oral glucocorticoids).

Examples of chronic inflammatory conditions that may be treated in the methods of the disclosure include, but are not limited to, rheumatoid arthritis, asthma, chronic obstructive pulmonary disease (COPD), osteoarthritis, rheumatic fever, allergic rhinitis, systemic lupus erythematosus, Crohn's disease, inflammatory bowel disease (IBD), ulcerative colitis, multiple sclerosis (MS), and psoriasis.

In certain embodiments of the methods of the disclosure, the chronic inflammatory disease or disorder is rheumatoid arthritis.

The present disclosure also provides improvements in preventing development of diabetes mellitus due to glucocorticoid-induced hyperglycemia in a subject in need of a treatment for a chronic inflammatory disease or disorder. Such methods include administering to the subject a therapeutically effective amount of AZD9567, or a salt, hydrate, solvate, or prodrug thereof, sufficient to control the glucocorticoid-induced hyperglycemia in the subject and prevent development of diabetes mellitus. In certain embodiments, the subject that can benefit from prevention of development of diabetes mellitus is a subject that is predisposed to type 2 diabetes mellitus.

In another aspect of the disclosure, the present disclosure provides methods of inhibiting progression of diabetes mellitus in a subject in need of a treatment for a chronic inflammatory disease or disorder and having type 2 diabetes mellitus. Such methods include: administering to the subject a therapeutically effective amount of AZD9567, or a salt, hydrate, solvate, or prodrug thereof, sufficient to control the glucocorticoid-induced hyperglycemia in the subject and inhibit progression of diabetes mellitus.

In certain embodiments of the methods of the disclosure, the subject has type 2 diabetes mellitus. For example, the subject with type 2 diabetes mellitus has HbAlc in the range of about 6.5% to about 9.5%, and/or fasting plasma glucose in the range of about 126 to about 220 mg/dL.

The subject may have type 2 diabetes mellitus for at least 6 months prior to administration of AZD9567, or a hydrate, solvate, or prodrug thereof. Such subject, in certain embodiments, received metformin therapy for at least 4 weeks, and optionally had HbAlc in the range of about 6% to about 9.5%. In certain embodiments, the subject received a combination of metformin therapy and sodium-glucose cotransporter 2 inhibitor (SGLT2i) or dipeptidyl peptidase-4 inhibitor (DPP4i) for at least 4 weeks, and optionally had HbAlc in the range of about 6% to about 8%. For example, the metformin therapy dosage may not change by more than 500 mg/day.

The subject of the disclosure as described herein, in certain embodiments, previously received oral corticosteroid therapy. In certain embodiments, the subject may develop diabetes mellitus following the oral corticosteroid therapy.

In certain embodiments of the methods of the disclosure, the subject is predisposed to type 2 diabetes mellitus (e.g., pre-diabetic). For example, the subject predisposed to type 2 diabetes mellitus has HbAlc in the range of 5.7% to 6.4%, and/or fasting plasma glucose in the range of about 100 to about 125 mg/dl, and/or is obese (Body Mass Index (BMI)≥30), overweight (BMI 25.0 to <30), has hypertension or has low high-density lipoprotein (HDL) with elevated triglycerides.

The subject of the disclosure as described herein, in certain embodiments, has or is predisposed to cardiovascular co-morbidities, such as lipotoxic cardiomyopathy or heart failure.

As provided above, 2,2-difluoro-N-[(1R,2S)-3-methyl-1-[1-(1-methyl-6-oxopyridin-3-yl) indazol-5-yl]oxy-1-phenylbutan-2-yl]propanamide (also known as AZD9567) or a salt, hydrate, solvate, or prodrug thereof is administered in the methods of the disclosure as described herein. AZD9567 is an investigational, oral, potent and selective nonsteroidal oral glucocorticoid receptor modulator developed for the treatment of inflammatory diseases.

AZD9567 is disclosed in International application publication No. WO 2016/046260 A1 (incorporated by reference herein in its entirety) as Example 1 and has the following structure:

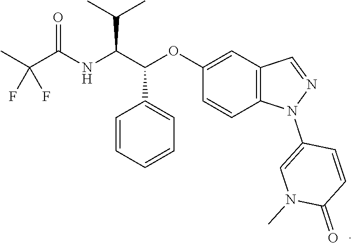 [SMILES: CC(C)[C@H](NC(=O)C(C)(F)F)[C@H](Oc1ccc2c(cnn2-c2ccc(=O)n(C)c2)c1)c1ccccc1]

AZD9567 has similar efficacy as oral steroids, such as prednisolone, but has a different binding mode to the glucocorticoid receptor from prednisolone. The anti-inflammatory effects of the two compounds are similar when assessed in vivo in a rat model of joint inflammation and ex vivo by inhibition of lipopolysaccharide (LPS)-stimulated tumour necrosis factor-α (TNFα) release in human whole blood. Unlike prednisolone, however, AZD9567 does not upregulate transcription of tyrosine aminotransferase (TAT), a key enzyme in gluconeogenesis known to be under direct control of the glucocorticoid receptor, in human hepatocytes in vitro. Furthermore, when the two compounds are coadministered in primary human hepatocytes, AZD9567 dose-dependently reduces prednisolone-induced upregulation of TAT transcription.

As used herein, the phrase “therapeutically effective amount” or “effective amount” refers to the amount of active compound or pharmaceutical agent that elicits the biological or medicinal response that is being sought in a tissue, system, animal, individual or human by a researcher, veterinarian, medical doctor or other clinician.

In certain embodiments, the therapeutically effective amount of AZD9567 reduces the subject's HbAlc by about 0.5% to about 1%. For example, in certain embodiments, the therapeutically effective amount of AZD9567 reduces the subject's HbAlc by about 0.6% to about 0.8%. In certain other embodiments, the therapeutically effective amount of AZD9567 reduces the subject's HbAlc by about 0.7%.

In certain embodiments of the methods of the disclosure as described herein, the therapeutically effective amount of AZD9567 lowers mean daily glucose increase in Mixed Meal Tolerance Test (MMTT), for example, as compared to prednisolone treatment. In certain embodiments, the therapeutically effective amount of AZD9567 lowers glucose area under the effect-time curve from time 0 to 4 hours (AUC₀₋₄ₕ) in MMTT, for example, as compared to prednisolone treatment.

In certain embodiments of the methods of the disclosure as described herein, the therapeutically effective amount of AZD9567 lowers glucagon AUC₀₋₄ₕ in MMTT, for example, as compared to prednisolone treatment. In certain embodiments, the therapeutically effective amount of AZD9567 lowers glucagon-like peptide-1 (GLP-1) AUC₀₋₄ₕ in MMTT, for example, as compared to prednisolone treatment. In certain embodiments, the therapeutically effective amount of AZD9567 improves insulin to glucagon ratio in MMTT, for example, as compared to prednisolone treatment.

In certain embodiments of the methods of the disclosure as described herein, the therapeutically effective amount of AZD9567 suppresses cortisol in MMTT, for example, as compared to prednisolone treatment.

In certain embodiments, an effective amount can be an amount suitable for
(i) inhibiting the progression the disease;
(ii) prophylactic use for example, preventing or limiting development of a disease, condition or disorder in an individual who may be predisposed or otherwise at risk to the disease, condition or disorder but does not yet experience or display the pathology or symptomatology of the disease;
(iii) inhibiting the disease; for example, inhibiting a disease, condition or disorder in an individual who is experiencing or displaying the pathology or symptomatology of the disease, condition or disorder;
(iv) ameliorating the referenced disease state, for example, ameliorating a disease, condition or disorder in an individual who is experiencing or displaying the pathology or symptomatology of the disease, condition or disorder (i.e., reversing or improving the pathology and/or symptomatology) such as decreasing the severity of disease; or
(v) eliciting the referenced biological effect.

Given the well-established clinical efficacy of prednisolone and its wide use, therapeutically effective amount of AZD9567 for treatment of chronic inflammatory disease or disorder, in certain embodiments, may be estimated to be an equipotent dose of AZD9567 based on prednisolone dose with the same anti-inflammatory effect. Methods for estimating the equipotent dose may be as described by Joachim Almquist et al. (CPT Pharmacometrics Syst. Pharmacol. (2020) 9, 444-455; doi: 10.1002/psp4.12536).

In certain embodiments of the method of the disclosure as described herein, the therapeutically effective amount of AZD9567 is in the range of about 20 to 80 mg per day. For example, in certain embodiments, the therapeutically effective amount of AZD9567 is in the range of about 30 to 80 mg per day, or about 40 to 72 mg per day, or about 30 to 50 mg per day, or about 60 to 80 mg per day.

In certain embodiments, the therapeutically effective amount of AZD9567 is about 40 mg daily. In certain embodiments, the therapeutically effective amount of AZD9567 is about 72 mg daily.

Identifying a subject that can benefit from the methods of the disclosure as described herein, is also within the scope of this disclosure. Thus, in certain embodiments, the methods of the disclosure further comprise identifying the subject having type 2 diabetes mellitus as disclosed herein (e.g., prior to administration of AZD9567). In certain embodiments, the methods of the disclosure further comprise identifying the subject predisposed to develop type 2 diabetes mellitus as disclosed herein (e.g., prior to administration of AZD9567). As noted above, such subject may have or be predisposed to cardiovascular co-morbidities, such as lipotoxic cardiomyopathy or heart failure.

### Example

The methods of the disclosure are illustrated further by the following example, which is not to be construed as limiting the disclosure in scope or spirit to the specific procedures and compounds described in them.

### Methods

Patient population. In clinical trial NCT04556760, eligible patients were 18 to 75 years old. Eligible patients had diagnosis of type 2 diabetes mellitus for 6 months prior to screening, such as HbAlc in the diabetes range or fasting plasma glucose 126-220 mg/dL. Eligible patients were on stable metformin therapy for at least 4 weeks, where no significant dose change (increase or decrease≥500 mg/day) has occurred prior to screening and HbAlc 6%-9.5%, or on dual therapy with metformin in combination with SGLT2i or DPP4i and HbAlc 6%-8%. Participants on dual therapy required 2 weeks wash-out of SGLT2i or DPP4i.

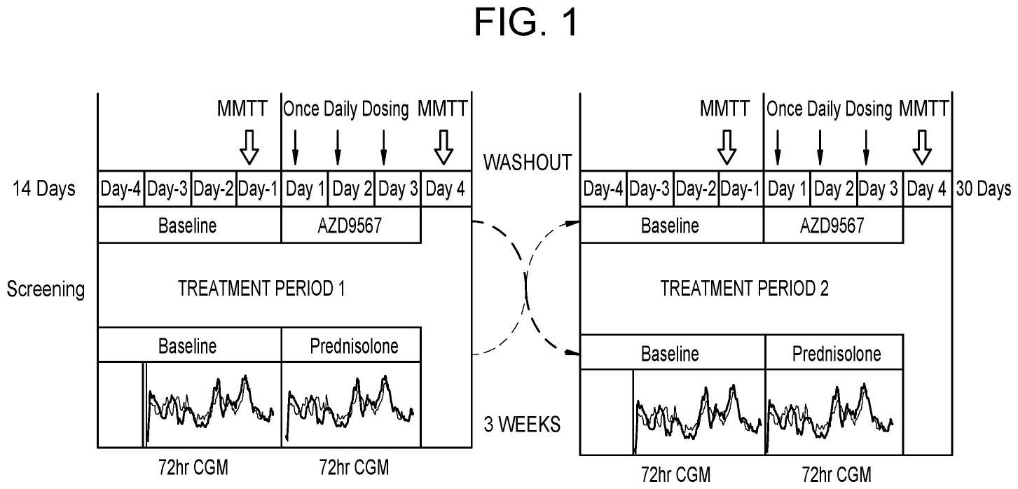

Study design. This was a randomized, double blind, multi-center, double dummy, and two-way cross-over study. The aim of the study was to compare the effect on glycaemic control and incretin response at equipotent doses of AZD9567 and prednisolone in diabetes patients. The AZD9567 and prednisolone doses were predicted to be equipotent, based on an ex vivo efficacy analysis from previous phase 1 studies. The study consisted of a total of three cohorts. Each cohort was treated for two 72-hour periods in a cross-over design, with a 3-week washout period between treatment periods as illustrated in FIG. 1. The total length of participant engagement (from screening to follow-up) is 79 days.

Cohort 1 included 27 patients. The patents were randomized in a ratio of 1:1 to receive 72 mg/day (oral suspension) AZD9567 and 40 mg/day prednisolone over two 72 hour periods in a cross over design: 72 mg AZD9567 for 3 consecutive days of each treatment period followed by 40 mg prednisolone for 3 consecutive days of each treatment period [AB sequence group], or 40 mg prednisolone for 3 consecutive days of each treatment period followed by 72 mg AZD9567 for 3 consecutive days of each treatment period [BA sequence group].

Cohort 2 included 8 patients. The patents were randomized in a ratio of 1:1 to receive 40 mg/day (oral suspension) AZD9567 and prednisolone over two 72 hour periods in a cross over design: 40 mg AZD9567 for 3 consecutive days of each treatment period followed by 20 mg prednisolone for 3 consecutive days of each treatment period [AB sequence group] or 20 mg prednisolone for 3 consecutive days of each treatment period followed by 40 mg AZD9567 for 3 consecutive days of each treatment period [BA sequence group].

Cohort 3 included 9 patients. The patents were randomized in a ratio of 1:1 to receive placebo and 5 mg/day prednisolone over two 72 hour periods in a cross over design: placebo for 3 consecutive days of each treatment period followed by 5 mg prednisolone for 3 consecutive days of each treatment period [AB sequence group] or 5 mg prednisolone for 3 consecutive days of each treatment period followed by placebo for 3 consecutive days of each treatment period [BA sequence group]).

Several methods are available to assess glucose homeostasis, and provide different insights into glycaemic physiology: mixed meal tolerance test (MMTT), continuous glucose monitoring (CGM), and oral glucose tolerance test (OGTT).

MMTT provides a fixed composition of protein, carbohydrates and fat, and is meant to mimic “normal” meal. It generally consists of 30.2 g protein, 19.6 g of fat, 75 g of carbohydrates with 6.6. g of fiber, totaling 612 kCal. It requires a standardized dinner the night before the test, 12 h fasting prior to the test, and standardized breakfast consumed (<30 min) at 7 AM (solid meal) consisting of pre-determined quantities of carbohydrates, proteins and fats. Sampling is done at −15, 10, 20, 30, 60, 75, 120, 180, and 240 min.

CGM includes a normal diet and is reflective of daily glucose control under “real world” conditions.

The primary endpoint was change in glucose AUC (0-4 h) versus baseline compared to prednisolone following a standardized mixed meal tolerance test (MMTT) on Days −1, 4, 27, and 31. The change from baseline in glucose AUC (0-4 h) was analyzed using a mixed model repeated measures (MMRM) with baseline included as covariate.

The secondary endpoints evaluated included:
mean daily glucose at 48-72 hours treatment as determined from multiple measures via the Continuous Glucose Monitoring (CGM) system on days-2, 3, 26 and 30 and as analyzed using a MMRM analysis with baseline as covariate;
rise in mean daily glucose over 24-hour periods from start of IMP dosing (0-24 hours, 24-48 hours, 48-72 hours) on days 1, 2, 3, 28, 29, and 30 and as analyzed using an MMRM analysis with baseline as covariate;
change from baseline in fasting glucose on days-1, 4, 27, and 31; change from baseline AUC (0-4) on hormones related to glucose homeostasis (such as insulin, glucagon, GLP-1 and GIP following MMTT) on days-1, 4, 27, and 31;
change from baseline in AUC (0-4) on C-peptide on days-1, 4, 27, and 31; and MMTT derived first phase insulin response on days-1, 4, 27, and 31;
24-hour potassium concentration (on days-1, 3, 27 and 30), as measured in urine over 24 hours;
24-hour sodium concentration (on days-1, 3, 27 and 30), as measured in urine over 24 hours;
area under the plasma concentration versus time curve from zero to the last quantifiable concentration (AUClast) on days 3, 4, 30, and 31 (Pre-dose, Post-dose 0.25, 0.5, 1, 1.5, 2, 3, 4, 6, 8, 12, 24, 30 hours), as derived using standard non-compartmental methods using WinNonLin version 8.1 or higher (Certara);
area under the plasma concentration versus time curve from zero to 24 hours post-dose (AUC (0-24)) on days 3, 4, 30, and 31 (Pre-dose, Post-dose 0.25, 0.5, 1, 1.5, 2, 3, 4, 6, 8, 12, 24, 30 hours), as derived using standard non-compartmental methods;
area under the plasma concentration versus time curve from zero to 6 hours post-dose (AUC (0-6)) on days 3, 4, 30, and 31 (Pre-dose, Post-dose 0.25, 0.5, 1, 1.5, 2, 3, 4, 6, 8, 12, 24, 30 hours), as derived using standard non-compartmental methods;
maximum observed drug concentration (Cₘₐₓ) on days 3, 4, 30, and 31 (Pre-dose, Post-dose 0.25, 0.5, 1, 1.5, 2, 3, 4, 6, 8, 12, 24, 30 hours), as derived using standard non-compartmental methods;
time to reach maximum observed drug concentration (tₘₐₓ) on days 3, 4, 30, and 31 (Pre-dose, Post-dose 0.25, 0.5, 1, 1.5, 2, 3, 4, 6, 8, 12, 24, 30 hours), as derived using standard non-compartmental methods;
terminal elimination half-life (t½λz) on days 3, 4, 30, and 31 (Pre-dose, Post-dose 0.25, 0.5, 1, 1.5, 2, 3, 4, 6, 8, 12, 24, 30 hours), as derived using standard non-compartmental methods;
apparent total body clearance of drug from plasma after extravascular (CL/F) on days 3, 4, 30, and 31 (Pre-dose, Post-dose 0.25, 0.5, 1, 1.5, 2, 3, 4, 6, 8, 12, 24, 30 hours), as derived using standard non-compartmental methods;
apparent volume of distribution following extravascular administration (Vz/F) on days 3, 4, 30, and 31 (Pre-dose, Post-dose 0.25, 0.5, 1, 1.5, 2, 3, 4, 6, 8, 12, 24, 30 hours), as derived using standard non-compartmental methods;
TNFα concentrations on days 3 and 30 (Pre-dose, Post-dose 1, 2, 4, 8, 12, and 24 hours) (e.g., assessing the relationship between AZD9567 exposure and inhibition of LPS-stimulated TNFα release for high and low dose comparison (Cohorts 1 and 2));
change in free fatty acids following a MMTT on days-1, 4, 27, and 31;
homeostatic model assessment-insulin resistance (HOMA-IR) on days-1, 4, 27, and 31, on derived measures of beta cell function from the MMTT;
HOMA-insulin sensitivity on days-1, 4, 27, and 31, on derived measures of beta cell function from the MMTT; and
safety and tolerability of AZD9567 by assessing the number of participants with adverse events (AEs) from screening up to 79 days including AEs/severe AEs (SAEs), vital signs, electrocardiograms (ECGs), changes in clinical chemistry/haematology parameters, morning serum cortisol, and adrenocorticotropic hormone.

### Results

An interim analysis following completion of Cohort 1 (high dose) has been conducted to review high level data. As noted above, glycaemic control was evaluated by two methods: CGM (throughout the three days of dosing) and MMTT (at baseline pre-dose, and 24 hours after last dose).

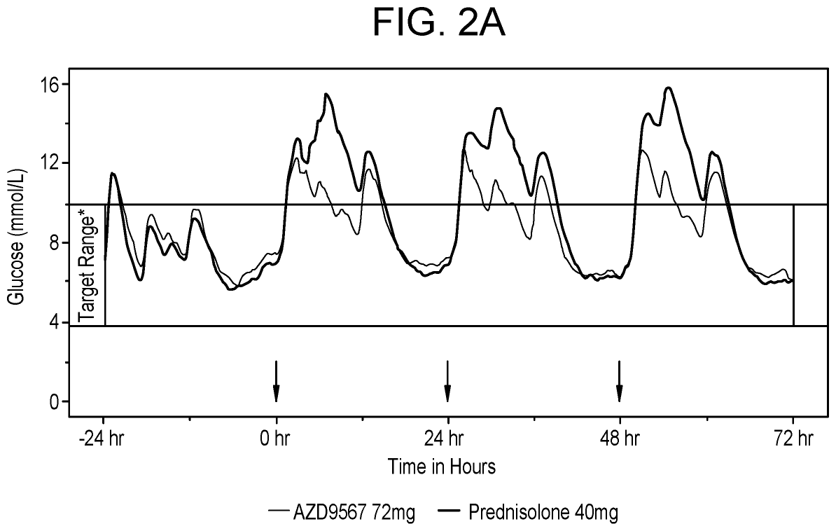

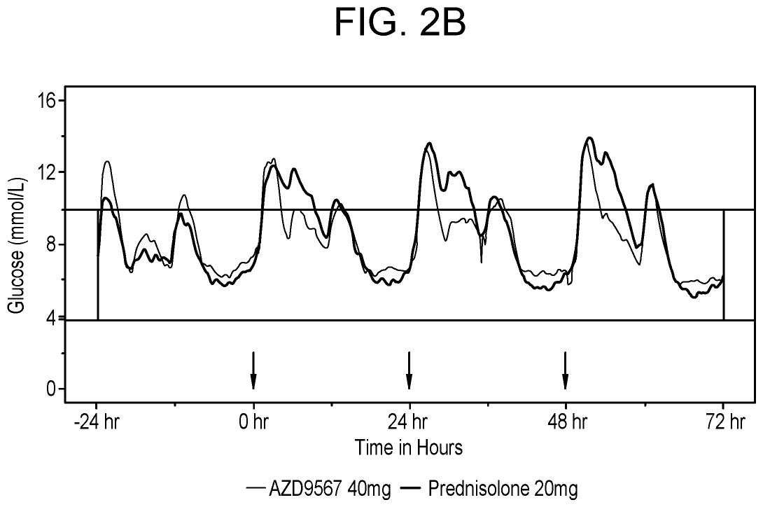

As provided in FIG. 2A, CGM demonstrated clear and consistent differentiation on glycaemic control for AZD9567 versus prednisolone in Cohort 1, with the magnitude of difference predicted to translate to a clinically meaningful reduction in HbAlc. Glycaemic target range set to 70-180 mg/dl (3.9-10.0 mmol/L) for patients with type 2 diabetes, ADA Standards of Medical Care in Diabetes [January 2020, Diabetes Care 43 (Supplement 1)]. The differentiation on glycaemic control for AZD9567 versus prednisolone was lower in Cohort 2.

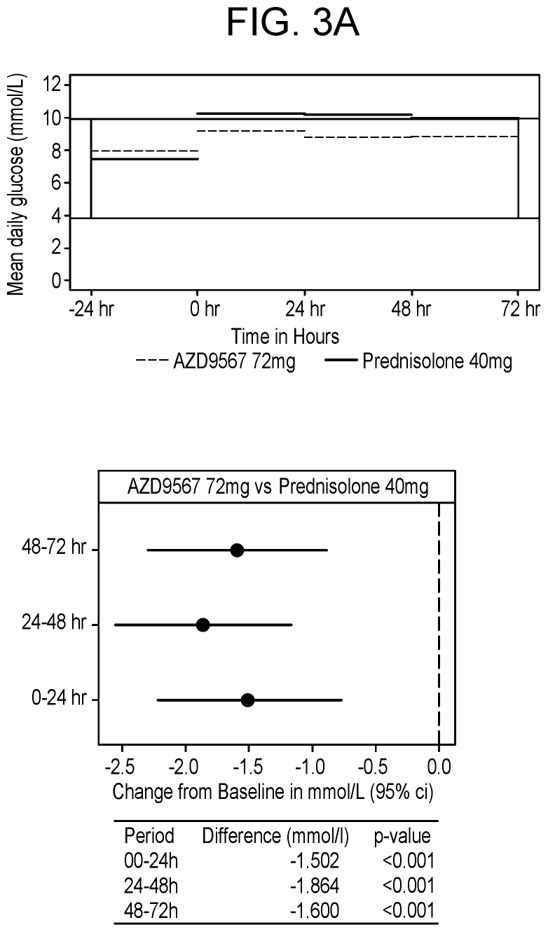

AZD9567 (Cohort 1) shows significantly lower mean daily glucose increase as compared to prednisolone (FIG. 3A). The increase in mean daily glucose induced by AZD9567 was within glycaemic targets for patients with type 2 diabetes mellius. Difference between AZD9567 and prednisolone at 48-72 h corresponds to an HbAlc difference of 0.7%, based on estimated Average Glucose (eAG) equation, in the given glycaemic range.

$$eAG ( mg / dl ) = 28 \text{.7} \times A 1 C - 46.7$$

AZD9567 lower dose (Cohort 2) shows lower mean daily glucose increase, corresponding to an HbAlc difference of 0.4% (based on eAG equation), in the given glycaemic range (FIG. 3B). Both of these results are predicted to translate to clinically meaningful change in HbAlc. No differentiation on glycaemic control was observed for low dose prednisolone vs placebo on CGM in Cohort 3 (results not shown).

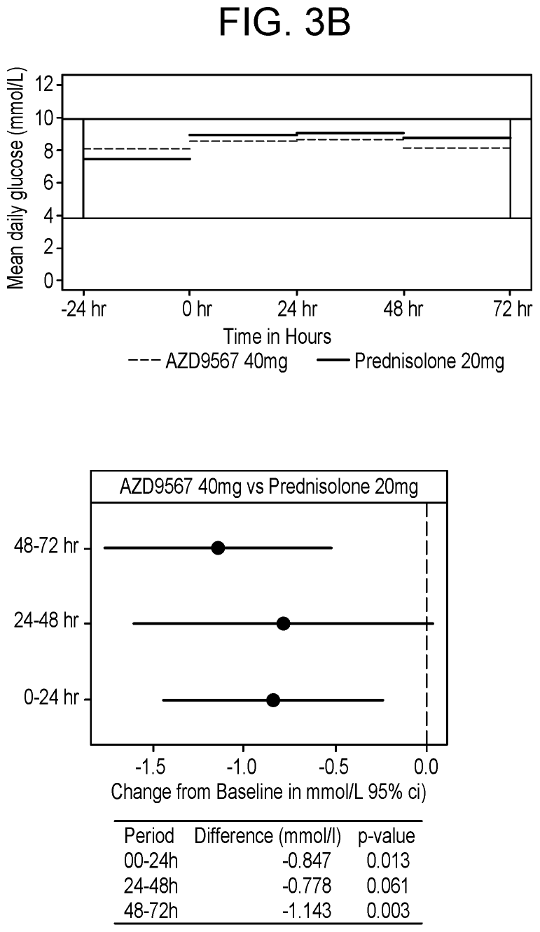

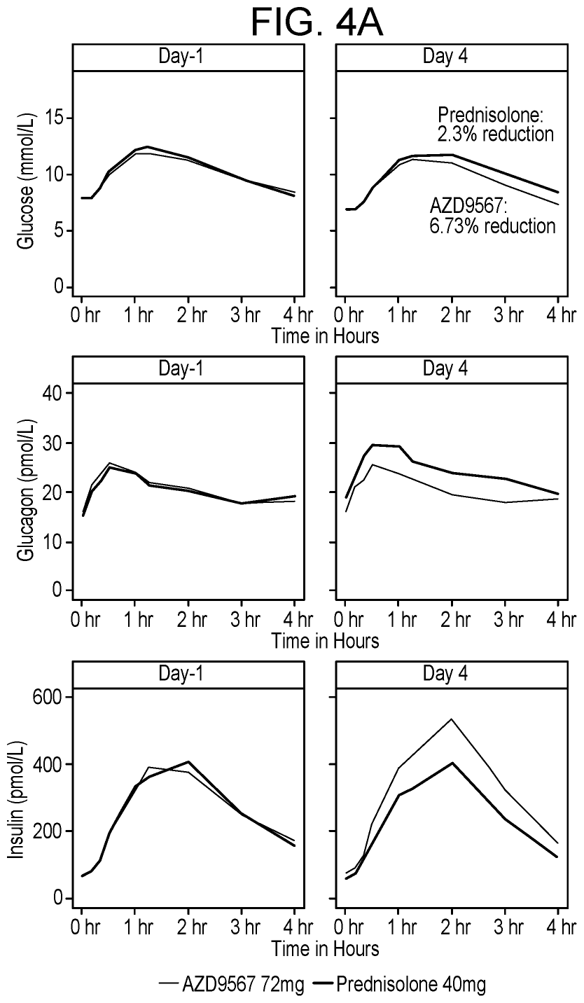

For the primary endpoint, there was favourable differentiation in glucose AUC₀₋₄ₕ following MMTT (24 h after last dose) in Cohort 1 (FIG. 4A). Statistically significant difference of −4.54 (95CI: 0.01, 8.88) % (p=0.05) in reduction of glucose AUC₀₋₄ₕ after MMTT was observed. Absolute difference in reduction of glucose AUC₀₋₄ₕ after MMTT was 132.95 (95% CI 9.40, 256.1) min*mmol/L (p=0.036). Glucagon secretion was unaffected by AZD9567 but increased with prednisolone. This result is significant because increased glucagon results in elevated hepatic glucose output, increased insulin resistance and beta cell strain. MMTT-stimulated insulin secretion increased with AZD9567 but was lower for prednisolone, leading to marked improvement in insulin:glucagon ratio with AZD9567. Marked differentiation in pancreatic beta cell response has possible translation to improved insulin secretion long-term. Based on these observed differences in glucagon and insulin, AZD9567 is predicted to engender slower progression of diabetes than prednisolone.

In addition, AZD9567 achieves prespecified CGM Go criteria vs prednisolone. Target Value (TV) was set at a difference of 1.221 mmol/L (about 0.8% HbAlc over 6 months); Lower Reference Value (LRV) was set at difference of 0.599 mmol/L (about 0.4% HbAlc over 6 months). Observed difference of 1.507 mmol/L at 48-72 hrs exceeds TV.

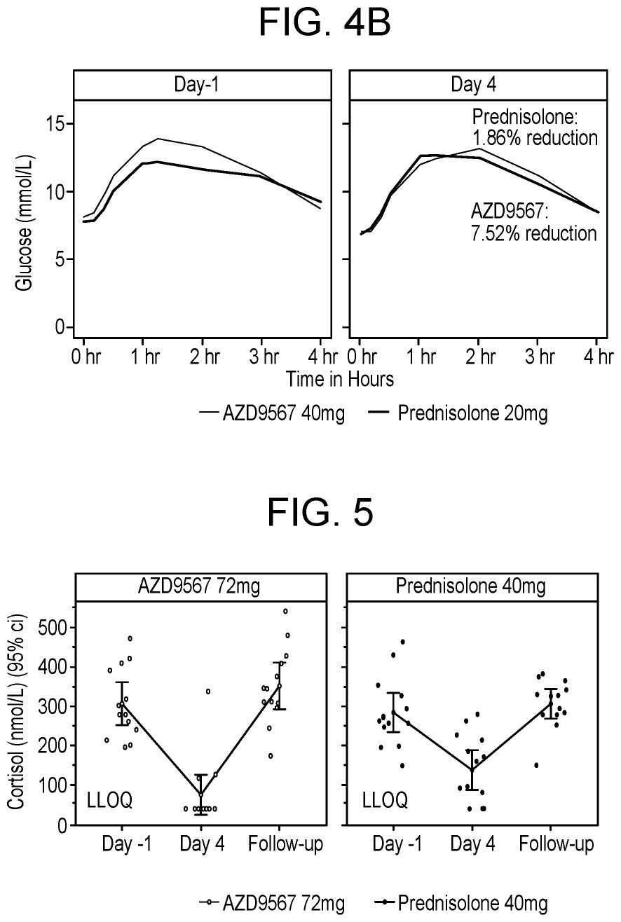

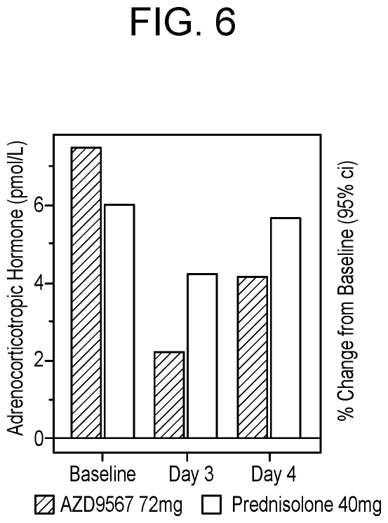

As shown in FIGS. 5 and 6, a marked, target-related, reversible cortisol suppression is observed with AZD9567 during treatment, returning to baseline at follow-up, with adrenocorticotropic hormone (ACTH) concentrations indicative of preserved hypothalamic-pituitary-adrenal (HPA) axis capacity. These results are in line with previous studies.

Lastly, higher systemic exposure observed for AZD9567 and prednisolone, and increased AZD9567 elimination half-life, in diabetic patients as compared to healthy volunteers in phase 1 study. No significant new safety concerns observed following the analysis of Cohort 1. For example, AZD9567 has lower rate of treatment intensification (i.e., dose increase of current oral antidiabetic therapy, or start/addition of insulin) as compared to prednisone in diagnosed diabetic patients. Also AZD9567 has lower incidence of progression to type 2 diabetes mellitus as compared to prednisone in population predisposed to type 2 diabetes mellitus Finally, AZD9567 has lower incidence of hospitalization vs prednisone in heart failure patients.

Thus, AZD9567 represents a novel alternative to oral corticosteroids in the treatment of chronic inflammatory conditions requiring medium to high dose (e.g., ≥10 mg prednisone, or equivalent nominal dose for other oral glucocorticoid molecules) of oral corticosteroids in patients with metabolic and cardiovascular co-morbidities, such as diabetes, predisposed to (i.e., at risk of) diabetes, and/or with heart failure.

It is understood that the examples and embodiments described herein are for illustrative purposes only and that various modifications or changes in light thereof will be suggested to persons skilled in the art and are to be incorporated within the spirit and purview of this application and scope of the appended claims. All publications, patents, and patent applications cited herein are hereby incorporated herein by reference for all purposes.

What is claimed is:

1. A method of treating a chronic inflammatory disease or disorder in a subject, wherein the subject has type 2 diabetes mellitus or the subject is predisposed to type 2 diabetes mellitus, the method comprising administering to the subject a therapeutically effective amount of 2,2-difluoro-N-[(1R,2S)-3-methyl-1-[1-(1-methyl-6-oxopyridin-3-yl) indazol-5-yl]oxy-1-phenylbutan-2-yl]propanamide (AZD9567), or a salt, hydrate, solvate, or prodrug thereof,
wherein the therapeutically effective amount of AZD9567 is sufficient to treat the chronic inflammatory disease or disorder in the subject, and
wherein the therapeutically effective amount of AZD9567 is sufficient to control glycaemia in the subject.

2. The method of claim 1, wherein the chronic inflammatory disease or disorder is responsive to oral corticosteroids.

3. The method of either claim 1 or claim 2, wherein the chronic inflammatory disease or disorder is selected from rheumatoid arthritis, asthma, chronic obstructive pulmonary disease (COPD), osteoarthritis, rheumatic fever, allergic rhinitis, systemic lupus erythematosus, Crohn's disease, inflammatory bowel disease (IBD), ulcerative colitis, multiple sclerosis (MS), and psoriasis.

4. The method of either claim 1 or claim 2, wherein the chronic inflammatory disease or disorder is rheumatoid arthritis.

5. A method of preventing development of diabetes mellitus due to glucocorticoid-induced hyperglycemia in a subject in need of a treatment for a chronic inflammatory disease or disorder, the method comprising administering to the subject a therapeutically effective amount of 2,2-difluoro-N-[(1R,2S)-3-methyl-1-[1-(1-methyl-6-oxopyridin-3-yl) indazol-5-yl]oxy-1-phenylbutan-2-yl]propanamide (AZD9567), or a salt, hydrate, solvate, or prodrug thereof,
wherein the therapeutically effective amount of AZD9567 is sufficient to control the glucocorticoid-induced hyperglycemia in the subject and prevent development of diabetes mellitus.

6. A method of inhibiting progression of diabetes mellitus in a subject in need of a treatment for a chronic inflammatory disease or disorder and having type 2 diabetes mellitus, the method comprising administering to the subject a therapeutically effective amount of 2,2-difluoro-N-[(1R,2S)-3-methyl-1-[1-(1-methyl-6-oxopyridin-3-yl) indazol-5-yl]oxy-1-phenylbutan-2-yl]propanamide (AZD9567), or a salt, hydrate, solvate, or prodrug thereof,
wherein the therapeutically effective amount of AZD9567 is sufficient to control the glucocorticoid-induced hyperglycemia in the subject and inhibit progression of diabetes mellitus in the subject.

7. The method of any one of claims 1 to 6, wherein the subject has type 2 diabetes mellitus.

8. The method of claim 7, wherein the subject has diabetes mellitus for at least 6 months prior to administration of AZD9567, or a hydrate, solvate, or prodrug thereof.

9. The method of either claim 7 or claim 8, wherein the subject has HbAlc in the range of about 6.5% to about 9.5%, and/or fasting plasma glucose in the range of about 126 to about 220 mg/dL.

10. The method of claim 9, wherein the subject received metformin therapy for at least 4 weeks, and optionally had HbAlc in the range of about 6% to about 9.5%.

11. The method of claim 9, wherein the subject received a combination of metformin therapy and sodium-glucose cotransporter 2 inhibitor (SGLT2i) or dipeptidyl peptidase-4 inhibitor (DPP4i) for at least 4 weeks, and optionally had HbAlc in the range of about 6% to about 8%.

12. The method of either claim 10 or claim 11, wherein the metformin therapy dosage does not change by more than 500 mg/day.

13. The method of any one of claims 1 to 6, wherein the subject is predisposed to type 2 diabetes mellitus, and optionally to lipotoxic cardiomyopathy or heart failure.

14. The method of claim 13, wherein the subject has HbAlc in the range of 5.7% to 6.4%, and/or fasting plasma glucose in the range of about 100 to about 125 mg/dL.

15. The method of any one of claims 1 to 14, wherein the subject previously received oral corticosteroid therapy.

16. The method of claim 15, wherein the subject develops diabetes mellitus following the oral corticosteroid therapy.

17. The method of any one of claims 1 to 16, wherein the therapeutically effective amount of AZD9567 reduces HbAlc by about 0.5% to about 1%.

18. The method of any one of claims 1 to 16, wherein the therapeutically effective amount of AZD9567 reduces HbAlc by about 0.6% to about 0.8%.

19. The method of claim 18, wherein the therapeutically effective amount of AZD9567 reduces HbAlc by about 0.7%.

20. The method of any one of claims 1 to 19, wherein the therapeutically effective amount of AZD9567 lowers mean daily glucose increase in Mixed Meal Tolerance Test (MMTT) (for example, as compared to prednisolone treatment).

21. The method of any one of claims 1 to 20, wherein the therapeutically effective amount of AZD9567 lowers glucose AUC₀₋₄ₕ in MMTT (for example, as compared to prednisolone treatment).

22. The method of any one of claims 1 to 21, wherein the therapeutically effective amount of AZD9567 lowers glucagon AUC₀₋₄ₕ in MMTT (for example, as compared to prednisolone treatment).

23. The method of any one of claims 1 to 22, wherein the therapeutically effective amount of AZD9567 lowers glucagon-like peptide-1 (GLP-1) AUC₀₋₄ₕ in MMTT (for example, as compared to prednisolone treatment).

24. The method of any one of claims 1 to 22, wherein the therapeutically effective amount of AZD9567 improves insulin to glucagon ratio in MMTT (for example, as compared to prednisolone treatment).

25. The method of any one of claims 1 to 24, wherein the therapeutically effective amount of AZD9567 suppresses cortisol in MMTT (for example, as compared to prednisolone treatment).

26. The method of any one of claims 1 to 25, wherein the therapeutically effective amount of AZD9567 is in the range of about 20 to 80 mg per day.

27. The method of any one of claims 1 to 25, wherein the therapeutically effective amount of AZD9567 is in the range of about 40 to 72 mg per day.

28. The method of any one of claims 1 to 25, wherein the therapeutically effective amount of AZD9567 is about 72 mg daily.

29. The method of any one of claims 1 to 25, wherein the therapeutically effective amount of AZD9567 is about 40 mg daily.

30. The method of any one of claims 1 to 29, further comprising identifying the subject having type 2 diabetes mellitus.

31. The method of claim 30, wherein the subject has HbAlc of at least 6.5% or higher.

32. The method of any one of claims 1 to 29, further comprising identifying the subject predisposed to develop type 2 diabetes mellitus.

33. The method of claim 32, wherein the subject has HbAlc in the range of 5.7% to 6.4%.

34. The method of claim 31, wherein the subject is predisposed to develop lipotoxic cardiomyopathy or heart failure.
